(19) United States
(12) Patent Application Publication
(10) Pub. No.: US 2007/0028926 A1
(43) Pub. Date: Feb. 8, 2007

(54) Snore and sleep apnea syndrome medical treatment device

(76) Inventor: Hiroo Kotani, Hiroshima (JP)

Correspondence Address: FLYNN THIEL BOUTELL & TANIS, P.C., 2026 RAMBLING ROAD, KALAMAZOO, MI, 49008-1631, US

(21) Appl. No.: 10/572,441

(22) PCT Filed: Sep. 13, 2004
(86) PCT No.: PCT/JP04/13304

§ 371 Date: Mar. 17, 2006

(30) Foreign Application Priority Data
Sep. 22, 2003 (JP) 2003-329791

(51) Int. Cl. A61F 5/56

(52) U.S. Cl. 128/848

(57) ABSTRACT

It is an object of this invention to provide a medical treatment device for curing snore and sleep palate syndrome, which is small in size, easy to place, allows patients to have communication after placement, does not induce the patients any physical disorder to jaw joint and jaw muscle, simple in operation and structure, and highly effective in physical treatment. There is provided a medical treatment device for curing snore and sleep apnea syndrome, which comprises a fitting body 10 and a guide plate 20. The fitting body 10 is formed in a generally U-shape in plane view and in cross section, and it is fitted to the teeth 3 and the gingiva 5 of the upper jaw 1. The guide plate 20 is extended in the diagonal rear lower side direction from the front portion of the fitting body 10. The guide plate 20 makes a contact with front teeth 4a of a lower jaw 2 at an outer surface 21 thereof, and it forwards the front teeth 4a of the lower jaw 2 to take a forwarding position. The guide plate 20 makes a contact with a tongue 6 at an inner surface thereof for holding the tongue 6 in a forwarding position.

### TECHNICAL FIELD

This invention relates to a medical treatment device installed in an oral cavity to cure snore and sleep apnea syndrome.

### BACKGROUND OF THE INVENTION

The snore and the sleep apnea syndrome, each has been recognized as one of the modern disease, happen when the upper airway 8 is blocked by the tongue base 6a or by the soft palate 7 during sleep (See FIG. 1).

There have conventionally been provided some medical treatment devices for curing the snore and the sleep apnea syndrome, which are to be fitted in the oral cavity of a patient. Among those, there is a type of device which is structured to forward the lower jaw of the patient (hereinafter called the “lower jaw forwarding device”), a type of device which is structured to hold the tongue (hereinafter called as the “tongue holding device”), and also a type of device which is structured to uplift the soft palate (hereinafter called as the “soft palate uplifting device”), as disclosed in Japanese Published Patent Application No. 2000-139984.

The lower jaw forwarding device holds the lower jawbone in a forwarding position to move the tongue bone and the structure of the tongue base, thereby enlarging the space of the upper airway. This type is commonly used in this kind of devices. Among the lower jaw forwarding device, there are one-piece type and two-piece type. The former one-piece type comprises an upper jaw member and a lower jaw member integrally formed, whereas the latter two-piece type comprises an upper jaw member and a lower jaw member separately formed. Both types are structured to fit both the upper and lower jaws, so that they are considerably large in size and are troublesome to install. Further, after it is installed, the devices occupy large volume in the oral cavity, so that it is hard for the patient to have communication.

Moreover, although the two-piece type may allow the patient to move his/her jaws a little easier compared to the one-piece type, the lower jaw is restricted to take and maintain a forwarding position all the time, so that it would bring the jaw joint and the jaw muscle some physical disorder.

Further, when adjusting the forwarding position of the lower jaw, there has been adopted a subjective method in which the forwarding position is set by observing the stomatognathic system of each patient, while not standardizing the setting method, so that there have been occurring some physical disorders in the stomatognathic system of patients. For that reason, after setting the forwarding position of the lower jaw, its position often has to be readjusted and the device is often required to be remade, thereby making the operation and the structure of the device more complicated.

The tongue holding device holds the tongue in a forwarding position, and the soft palate uplifting device uplifts the soft palate, both enlarging the space of the airway to prevent the snore and the sleep apnea syndrome.

### DISCLOSER OF THE INVENTION

### PROBLEMS TO BE SOLVED BY THE INVENTION

However, the above-mentioned three conventional devices are structured only to forward the lower jaw, to hold the tongue, and to uplift the soft palate, respectively, so that they are not effective enough to cure the snore and the sleep apnea syndrome.

This invention is invented to overcome those problems, and therefore it is an object of this invention to provide a medical treatment device for curing the snore and the sleep palate syndrome, which is small in size, easily installable, allows communication even after installation, unaffecting any physical disorder to jaw joint and jaw muscle, is simple in operation and structure, and is highly effective in cure treatment.

### MEANS TO SOLVE THE PROBLEMS

The invention will be described referring to FIGS. 2 to 6. A first aspect of the invention provides a medical treatment device for curing the snore and the sleep apnea syndrome, which comprises a fitting body 10 and a tongue piece-like guide plate 20. The fitting body 10 is formed in a generally U-shape in plane view and in cross section, and it is fitted to the teeth 3 and the gingiva 5 of the upper jaw 1. The guide plate 20 is extended in the diagonal rear lower direction from the front portion of the fitting body 10. The guide plate 20 makes a contact with front teeth 4a of a lower jaw 2 at an outer surface 21 thereof, and it forwards the front teeth 4a of the lower jaw 2 to take a forward posture. The guide plate 20 makes a contact with a tongue 6 at an inner surface thereof for holding the tongue 6 in a forward posture.

A second aspect of the invention provides a medical treatment device for curing the snore and the sleep apnea syndrome, which comprises a fitting body 10 and a tongue piece-like guide plate 20. The fitting body 10 is formed in a generally U-shape in plane view and in cross section, and it is fitted to the teeth 3 and the gingiva 5 of the upper jaw 1. The guide plate 20 is extended in the diagonal rear lower side direction from the front portion of the fitting body 10. The guide plate 20 makes a contact with front teeth 4a of a lower jaw 2 at an outer surface 21 thereof, and it forwards the front teeth 4a of the lower jaw 2 to take a forward posture. The guide plate 20 makes a contact with a tongue 6 at an inner surface thereof for holding the tongue 6 in a forward posture.

The fitting body 10 comprises a bite adjustment portion 11 which is formed by the bottom portion thereof. The attachment portions 12, 12 are formed by the side portions thereof. The bite adjustment portion 11 and the guide plate 20 are formed in color similar to the gingiva 5 and the tongue 6, or they are formed in transparent. The attachment portions 12, 12 are formed in transparent.

### EFFECTS OF THE INVENTION

According to the first aspect of the invention, the fitting body 10 is fitted to the teeth 3 and the gingiva 5 of the upper jaw 1, so that it is more compact compared to the conventional device which is structured to fit both the upper jaw 1 and the lower jaw 2, and that it is easy to install. Further, with the device in mouth, a patient can move his/her upper jaw 1 and lower jaw 2, so that he/she can have communication after installing the device. Further, the device does not cause any troubles to the jaw joint and the jaw muscle. Moreover, the device can easily be adjusted to the oral cavity of any patient by adjusting the ramp and the shape of the guide plate 20, so that it is unneeded to remake the device. That is, by adding or removing the resin of the outer surface of the guide plate 20, the forward posture can easily be adjusted. In many cases in the past, the devices have been adjusted to the patients without remaking them as a whole. In the conventional regular type of device comprising the upper jaw member and the lower jaw member integrally formed, the upper jaw member and the lower jaw member are required to be connected, and thus, once they are tightly connected, there have been observed in many cases that they were required to be remade rather than to be adjusted. The device according to the invention does not require such trouble operations, and it is simple in structure.

Further, the guide plate 20 enables to guide the lower jaw 2 to move into the forward posture while holding the tongue 6 in the forward posture, so that it is prevented that the tongue base 6a and the soft plate 7 block the upper airway 8. Therefore, the space of the upper airway 8 is successfully enlarged, and the snore and the sleep apnea syndrome can be effectively cured. Moreover, during sleep, a patient may sometimes open his/her mouth unconsciously. In such case, the guide plate 20 makes a contact with the front teeth 4a of the lower jaw 2, thereby restraining the mouth to open and effectively preventing the lower jaw 2 from moving backward.

According to the second aspect of the invention, the medical treatment device can perform effects similar to the first aspect of the invention. Further, in this device, the bite adjustment portion 11 of the fitting body 10 and the guide plate 20 are formed with color similar to that of the gingiva 5 and the tongue 6, or they are formed in transparent, while the attachment members 12, 12 are formed in transparent, so that the device is good in appearance. That is, the bite adjustment portion 11 and the guide plate 20 are visualized like the gingiva 5 and the tongue 6, while the teeth 3 are seen through the transparent attachment portion 12, so that the device seems to perfectly fit in the oral cavity and it looks natural.

### BRIEF DESCRIPTION OF THE DRAWINGS

FIG. 1 is a sectional view showing the upper airway being blocked;

FIG. 2 is a sectional view showing a medical treatment device according to an embodiment of the invention being installed in the oral cavity;

FIG. 3 is a side elevation view showing a medical treatment device according to an embodiment of the invention;

FIG. 4 is a front elevation view showing a medical treatment device according to an embodiment of the invention;

FIG. 5 is a plane view showing a medical treatment device according to an embodiment of the invention;

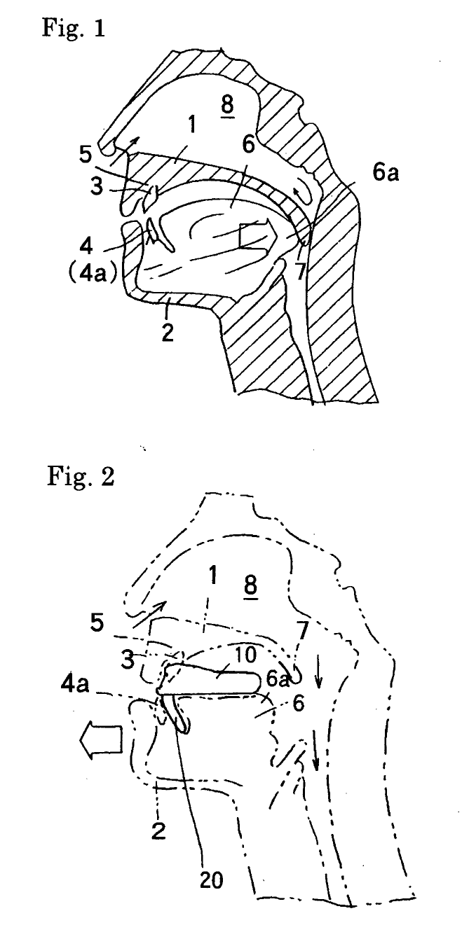

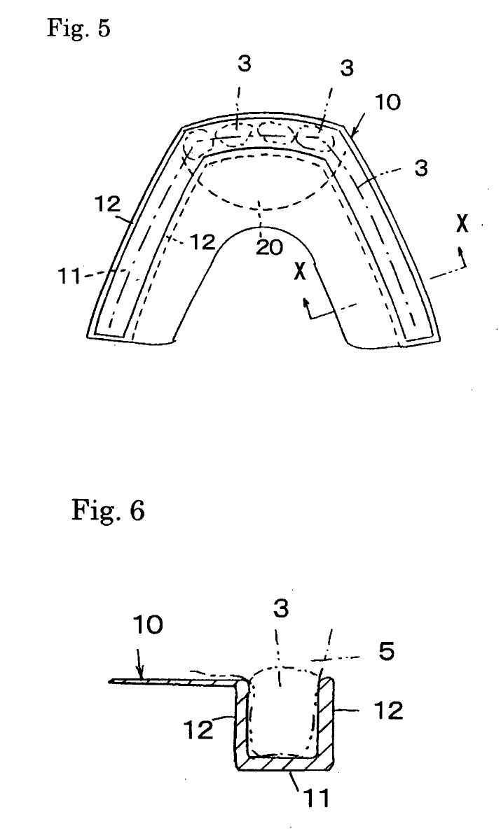

and, FIG. 6 is an enlarged sectional view along line X-X in FIG. 5.

### DESCRIPTION OF THE NUMERALS

1 upper jaw

2 lower jaw

3 teeth of upper jaw

4 teeth of lower jaw

4a front teeth of lower jaw

5 gingiva

6 tongue

6a tongue base

7 soft palate

8 upper airway

10 fitting body

11 bite adjustment portion

12 attachment portion

20 guide plate

21 outer surface

22 inner surface

### BEST MODE FOR CARRYING OUT THE INVENTION

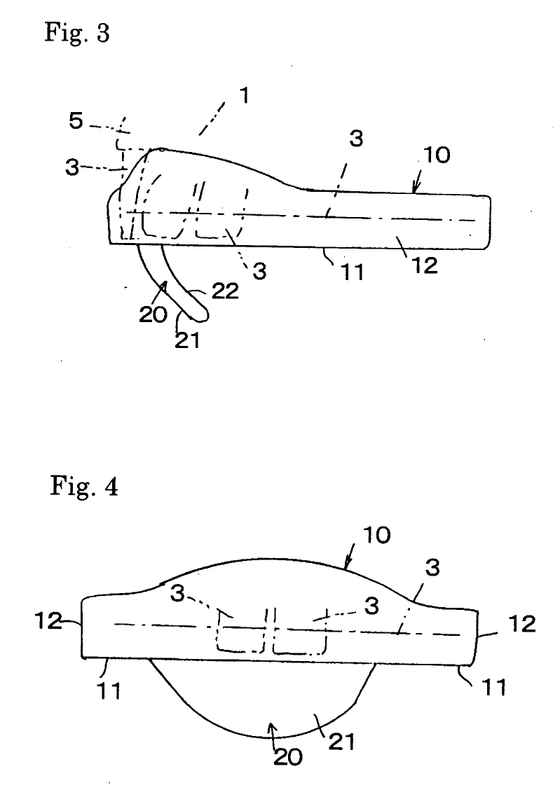

An embodiment of the medical treatment device for curing the snore and the sleep apnea syndrome according to the invention is illustrated in FIGS. 2 to 6. This device, being formed to be installed in the oral cavity in order to cure the snore and the sleep apnea syndrome, comprises a fitting body 10 and a tongue piece-like guide plate 20. The device is formed by thermoplastic resin which is good in workability. The device may be formed by thermoplastic resin and self curing resin, and it may also be formed by heat curing resin to improve its strength and durability.

The fitting body 10 is formed in a generally U-shape in plane view and in cross section, and it is fitted to all the teeth 3 and the gingiva 5 of the upper jaw 1. The guide plate 20 forms like a tongue shape being suspended in the diagonal rear lower side direction from the front portion (the portion of four front teeth 4a) of the fitting body 10. The outer surface 21 of the guide plate 20 makes a contact with the top end portion of the front teeth 4a of the lower jaw 2, guiding the front teeth 4a in a forwarding direction and guiding the lower jaw 2 in a position more front than its normal position. Further, the inner surface 22 of the guide plate 20 makes a contact with the tongue 6, holding the tongue 6 in between the teeth 3 of the upper jaw 1, thereby holding the tongue 6 in a forward posture.

The device can be installed by crowning the fitting body 10 on all the teeth 3 and the gingiva 5 of the upper jaw 1. Then, by moving the lower jaw 2 in a way of engaging the teeth 3, 4 of the upper jaw 1 with the lower jaw 2, the top end portion of the front teeth 4a of the lower jaw 2 is forced to make a contact with the outer surface 21 of the guide plate 20, and the front teeth 4a are guided in a forwarding direction. Therefore, the lower jaw 2 is forwarded to take a forward posture, making the tongue base 6a and the soft palate 7 not to block the upper airway 8, thereby enlarging the space of the upper airway 8. Further, by making the top end of the tongue 6 contact with the guide plate 20, positioning and holding the tongue 6 in between the cell of the oral cavity, the tongue 6 can be held in a forward posture, therefore, the space of the upper airway 8 can be enlarged as well. As a result of this, the snore and the sleep apnea syndrome can effectively cured.

The medical treatment device according to the embodiment of the invention is installed by crowning the fitting body 10 on the teeth 3 and the gingiva 5 of the upper jaw, so that unlike the conventional device which is fitted both the upper jaw 1 and the lower jaw 2, it can be formed in a compact size and it is easy to install. Further, the device is fitted only to the upper jaw 1, a patient can move both the upper jaw 1 and the lower jaw 2, so that the patient can have communication, and the device does not cause any trouble to the jaw joint and the jaw muscle. Moreover, the ramp, the shape, the thickness and the like of the guide plate 20 can be adjusted in order to adjust the forward posture, so that it is very seldom that the device has to be remade.

In the medical treatment device according to the embodiment of the invention, the lower portion of the fitting body 10 forms the bite adjusting portion 11 which stabilizes an engagement between the teeth 3 of the upper jaw 1 and the teeth 4 of the lower jaw 2, while both side portions of the fitting body 10 form the attachment portion 12. Further, the bite adjusting portion 11 and the guide plate 20 are formed in such color similar to that of the gingiva 5 and the tongue 6, or they are formed in transparent, while the attachment portions 12 are formed in transparent. Therefore, in a condition the device is installed in the oral cavity, the bite adjustment portion 11 and the guide plate 20 coordinate with the color of the gingiva 5 and the tongue 6, while the teeth 3 is visualized through the transparent attachment portion 12, which does not lead to visual uncomfortableness.

### PRACTICAL EXAMPLE

Table 1 and Table 2 show evaluations of effects in medical treatment devices installed in the oral cavity during sleep. Table 1 shows measurement results provided by a polysomnography, and Table 2 shows measurement results of lowest arterial oxygen saturation during sleep (LowestSp02) provided by an examination of arterial oxygen saturation in the blood.

In Table 1 and Table 2, “BMI” indicates fatness index (25.0 or more indicates fatness). “AHI” indicates Apnea-Hypopnca Index which includes the apnea and the hypopnca. The hypopnca is a condition in which amount of air ventilation decreases in half and the degree of oxygen saturation decreases 4% or more in a condition the air ventilation is not ceased. “AI” indicates Apnea Index showing a number of apnea in one hour.

TABLE 1

| | | | | AHI | | |
|---|---|---|---|---|---|---|
| | | | | Not | | Improvement |
| Patients | Age | Gender | BMI | worn | Worn | factor (%) |
| 1 | 70 | male | 27.7 | 30.2 | 7.5 | 75.2 |
| 2 | 68 | male | 21.8 | 38.6 | 4.2 | 89.1 |
| 3 | 37 | male | 27.1 | 10.0 | 6.2 | 38.0 |
| 4 | 30 | male | 30.4 | 42.0 | 18.3 | 56.4 |
| 5 | 63 | female | 26.1 | 24.4 | 19.3 | 20.9 |
| Average | 53.6 | | 26.6 | 29.0 | 11.1 | 55.9 |

| | AI | | | LowestSp02 | | |
|---|---|---|---|---|---|---|
| | Not | | Improvement | Not | | Improvement |
| Patients | worn | Worn | factor (%) | Worn | Worn | factor |
| 1 | 12.0 | 1.1 | 90.8 | 87 | 91 | 4.6 |
| 2 | 15.5 | 0.1 | 99.4 | 76 | 88 | 15.8 |
| 3 | 1.9 | 1.4 | 26.3 | 89 | 90 | 1.1 |
| 4 | 7.8 | 6.7 | 14.1 | 77 | 82 | 6.5 |
| 5 | 1.8 | 0.0 | 100.0 | 85 | 89 | 4.7 |
| Average | 7.8 | 1.9 | 66.1 | 82.8 | 88.0 | 6.5 |

TABLE 2

| | | | | LowestSp02 | | |
|---|---|---|---|---|---|---|
| | | | | Not | | Improvement |
| Patients | Age | Gender | BMI | worn | Worn | factor (%) |
| 6 | 31 | male | 28.7 | 79 | 81 | 2.5 |
| 7 | 38 | male | 27.1 | 86 | 88 | 2.3 |
| 8 | 58 | female | 25.0 | 75 | 85 | 13.3 |
| 9 | 78 | female | 14.8 | 69 | 83 | 20.3 |
| Average | 51.3 | | 23.9 | 77.3 | 84.3 | 9.6 |

1. A medical treatment device for curing sleep apnea syndrome comprising a fitting body (10) and a tongue piece-like guide plate (20) wherein,
said fitting body (10) being formed in a generally U-shape in plane view and in cross section for fitting the teeth (3) and the gingiva (5) of the upper jaw (1);
said guide plate (20) being extended in a diagonal rear lower side direction from the front portion of said fitting body (10);
said guide plate (20) making a contact with front teeth (4a) of a lower jaw (2) at an outer surface (21) thereof for forwarding said front teeth (4a) of said lower jaw (2) to take a forward posture;
and, said guide plate (20) making a contact with a tongue (6) at an inner surface thereof for holding said tongue (6) in a forward posture.

2. A medical treatment device for curing sleep apnea syndrome comprising a fitting body (10) and a tongue piece-like guide plate (20) wherein,
said fitting body (10) being formed in a generally U-shape in plane view and in cross section for fitting the teeth (3) and the gingiva (5) of the upper jaw (1);
said guide plate (20) being extended in a diagonal rear lower side direction from the front portion of said fitting body (10);
said guide plate (20) making a contact with front teeth (4a) of a lower jaw (2) at an outer surface (21) thereof for forwarding said front teeth (4a) of said lower jaw (2) to take a forward posture;
said guide plate (20) making a contact with a tongue (6) at an inner surface thereof for holding said tongue (6) in a forward posture;
said fitting body (10) comprising a bite adjustment portion (11) being formed by the bottom portion thereof, and attachment portions (12, 12) being formed by the side portions thereof;
said bite adjustment portion (11) and said guide plate (20) being formed in color similar to said gingiva (5) and said tongue (6) or formed in transparent;
and, said attachment portions (12, 12) being formed in transparent.
